(19) United States
(12) Patent Application Publication
(10) Pub. No.: US 2025/0332105 A1
(43) Pub. Date: Oct. 30, 2025

(54) AMORPHOUS SOLID DISPERSION COMPRISING (S)-1-(1-ACRYLOYLPYRROLIDIN-3-YL)-3-((3,5-DIMETHOXYPHENYL)ETHYNYL)-5-(METHYLAMINO)-1H-PYRAZOLE-4-CARBOXAMIDE

(71) Applicant: BEIJING INNOCARE PHARMA TECH CO., LTD., Beijing (CN)

(72) Inventor: Xiangyang CHEN, Beijing (CN)

(21) Appl. No.: 18/646,464

(22) Filed: Apr. 25, 2024

(51) Int. Cl. A61K 9/16; A61K 9/20; A61K 31/4155

(52) U.S. Cl. CPC A61K 9/1652; A61K 9/2054; A61K 31/4155

(57) ABSTRACT

The present invention relates to an amorphous solid dispersion comprises (S)-1-(1-acryloylpyrrolidin-3-yl)-3-((3,5-dimethoxyphenyl)ethynyl)-5-(methylamino)-1H-pyrazole-4-carboxamide (Compound I) and a pharmaceutically acceptable polymer. The present invention also relates to a pharmaceutical composition comprising the amorphous solid dispersion. The amorphous solid dispersion of the present invention is stable upon storage and provides better solubility and bioavailability when comparing with a crystalline form.

### CROSS-REFERENCE TO RELATED APPLICATION(S)

This application is a continuation of PCT/CN2022/128315, filed Oct. 28, 2022; which claims the benefit of U.S. Provisional Application No. 63/275,076, filed Nov. 3, 2021. The contents of the above-identified applications are incorporated herein by reference in their entirety.

### TECHNICAL FILED

The present invention relates to amorphous solid dispersions of (S)-1-(1-acryloylpyrrolidin-3-yl)-3-((3,5-dimethoxyphenyl)ethynyl)-5-(methylamino)-1H-pyrazole-4-carboxamide.

### BACKGROUND OF THE INVENTION

(S)-1-(1-acryloylpyrrolidin-3-yl)-3-((3,5-dimethoxyphenyl)ethynyl)-5-(methylamino)-1H-pyrazole-4-carboxamide (Compound I) is a potent inhibitor of fibroblast growth factor receptors (FGFR). The preparation of Compound I and its use in the treatment of cancers are described in WO2018/049781, which is incorporated herein by reference in its entirety.

### DESCRIPTION OF THE DRAWING

FIG. 1 shows the solubility profiles of Type A and ASD in a pH 6.8 phosphate buffer (n=3).

FIG. 2 shows the DSC diagrams of amorphous solid dispersions made via spray drying.

FIG. 3 shows the DSC diagrams of amorphous solid dispersions made via hot melt extrusion.

FIG. 4 shows the XRPD diagrams of Type A and amorphous solid dispersions (HPMCAS) via made by spray drying.

FIG. 5 shows the XRPD diagrams of Type A and amorphous solid dispersions (PVPVA) made via hot melt extrusion.

FIG. 6 shows the XRPD diagrams of HPMCAS-10-SDD after storage under (A) 25° C./60% RH, sealed and (B) 40° C./75% RH, sealed.

FIG. 7 shows the XRPD diagrams of PVPVA-10-HME after storage under 25° C./60% RH, sealed.

FIG. 8 shows the DSC diagrams of HPMCAS-10-SDD after storage under (A) 25° C./60% RH, sealed and (B) 40° C./75% RH, sealed.

FIG. 9 shows the DSC diagrams of PVPVA-10-HME after storage under 25° C./60% RH, sealed.

FIG. 10 shows the solubility profiles of HPMCAS-10-SDD after storage under (A) 25° C./60% RH, sealed and (B) 40° C./75% RH, sealed.

FIG. 11 shows the solubility profiles of PVPVA-10-HME after storage under 25° C./60% RH, sealed.

### DETAILED DESCRIPTION OF THE INVENTION

The present invention is directed to an amorphous solid dispersion (ASD) comprising (S)-1-(1-acryloylpyrrolidin-3-yl)-3-((3,5-dimethoxyphenyl)ethynyl)-5-(methylamino)-1H-pyrazole-4-carboxamide (Compound I), or a pharmaceutically acceptable salt thereof, and a pharmaceutically acceptable polymer.

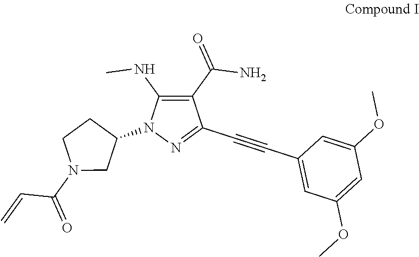 [SMILES: C=CC(=O)N1CC[C@H](n2nc(C#Cc3cc(OC)cc(OC)c3)c(C(N)=O)c2NC)C1]

The present invention discloses that the ASD form of Compound I has advantages over the crystalline form for use in preparing drug formulations. The ASD form of Compound I has good solubility and bioavailability, and is chemically and physically stable.

### Type A Crystalline Form

Type A crystalline form is prepared from Compound I as described in WO2018/049781. Type A crystalline form can be prepared by dissolving Compound I in acetone/water, followed by precipitation at a lower temperature.

The X-ray powder diffraction (XRPD) data of Type A are shown in Table 1.

TABLE 1

| 2-theta | d spacing | Intensity (%) |
|---|---|---|
| 6.66 | 13.26 | 19.2 |
| 8.69 | 10.17 | 0.6 |
| 10.05 | 8.79 | 16.3 |
| 10.96 | 8.06 | 33.9 |
| 12.62 | 7.01 | 0.8 |
| 13.29 | 6.65 | 5.1 |
| 13.99 | 6.33 | 40.8 |
| 14.55 | 6.08 | 0.5 |
| 15.71 | 5.64 | 14.3 |
| 16.14 | 5.49 | 17.1 |
| 17.42 | 5.09 | 67.0 |
| 17.82 | 4.97 | 41.1 |
| 18.36 | 4.83 | 4.0 |
| 19.90 | 4.46 | 17.6 |
| 20.29 | 4.37 | 3.1 |
| 20.63 | 4.30 | 3.3 |
| 21.58 | 4.11 | 10.9 |
| 22.14 | 4.01 | 100.0 |
| 22.85 | 3.89 | 16.2 |
| 23.40 | 3.80 | 18.7 |
| 24.07 | 3.69 | 29.8 |
| 24.91 | 3.57 | 22.9 |
| 25.43 | 3.50 | 4.1 |
| 26.37 | 3.38 | 5.0 |
| 26.74 | 3.33 | 7.2 |
| 27.48 | 3.24 | 4.9 |
| 28.28 | 3.15 | 55.5 |
| 29.74 | 3.00 | 2.3 |
| 30.50 | 2.93 | 10.3 |
| 31.86 | 2.81 | 6.0 |
| 32.35 | 2.77 | 4.6 |
| 33.27 | 2.69 | 1.3 |
| 33.88 | 2.64 | 3.4 |
| 34.53 | 2.60 | 3.6 |
| 35.17 | 2.55 | 8.2 |
| 35.55 | 2.52 | 1.8 |
| 36.04 | 2.49 | 1.4 |
| 36.61 | 2.45 | 2.2 |
| 36.83 | 2.44 | 2.0 |
| 37.51 | 2.40 | 1.8 |
| 38.64 | 2.33 | 5.9 |

Type A of the present invention has a solubility of 6.4 μg/mL after equilibrium in water at room temperature for 24 hours.

### Amorphous Solid Dispersion

The ASD of Compound I comprises Compound I or a pharmaceutically acceptable salt thereof, and a pharmaceutically acceptable polymer. The ASD of Compound I is stable in an amorphous form in a solid state for extended periods of time that may be used for preparing drug formulations. The ASD of Compound I has a desirable pharmaceutical profile, and it is amenable to manufacturing.

A pharmaceutically acceptable polymer is included in ASD to stabilize the compound and the dispersion, which may be a hydrophilic polymer, including cellulose based polymers (e.g., hydroxypropylmethyl cellulose (HPMC, hypromellose), ethyl cellulose, hydroxyethyl cellulose, hydroxyethylmethyl cellulose, hydroxypropyl cellulose, hypromellose phthalate (HPMCP), cellulose acetate, cellulose acetate phthalate, methyl cellulose, cellulose, carboxymethyl cellulose, microcrystalline cellulose, silicified microcrystalline cellulose, etc.), starch based polymers (e.g., hydroxypropyl starch, starches (including starches from any source, such as corn, potato, rice, wheat, which can be fully pregelatinized and partially gelatinized)), polyethylene glycol, polyacrylic acid, polyacrylamide, polyethylene oxide, polyvinylpyrrolidone, polyvinylalcohol, polyglycolized glycerides, polymethacrylates, hydrocolloids (e.g., carrageenan, chitosan, alginic acid, hyaluronic acid, pectinic acid, etc.).

Preferred polymers of the present invention include hydroxypropylmethylcellulose-acetate succinate (HPMC-AS), polyvinylpyrrolidone-vinyl acetate copolymer (PVP-VA), polyvinylpyrrolidone (PVP), and hydroxypropylmethylcellulose (HPMC). HPMC-AS and PVP-VA are further preferred.

The amount of Compound I in ASD is in general 5-60%, or 5-50%, 5-40%, 10-50%, or 10-40% by weight. For example, the amount of Compound I in ASD (drug loading) is 10% or 20% by weight.

The weight ratio of Compound I to the pharmaceutically acceptable polymer (e.g., HPMC-AS or PVP-VA) is in general in the range of 1:1 to 5:95, or 1:1 to 1:9.

For example, the ASD of Compound I comprises 5-40% w/w of Compound I and 60-95% w/w of HPMC-AS.

For example, the ASD of Compound I comprises 10-40% w/w of Compound I and 60-90% w/w of HPMC-AS.

For example, the ASD of Compound I comprises 5-30% w/w of Compound I and 70-95% w/w of HPMC-AS.

For example, the ASD of Compound I comprises 10-30% w/w of Compound I and 70-90% w/w of HPMC-AS.

For example, the ASD of Compound I comprises 5-25% w/w of Compound I and 75-95% w/w of HPMC-AS.

For example, the ASD of Compound I comprises 10-25% w/w of Compound I and 75-90% w/w of HPMC-AS.

For example, the ASD of Compound I comprises 5-20% w/w of Compound I and 80-95% w/w of HPMC-AS.

For example, the ASD of Compound I comprises 10-20% w/w of Compound I and 80-90% w/w of HPMC-AS.

For example, the ASD of Compound I comprises 10-40% w/w of Compound I and 60-90% w/w of PVP-VA.

In one preferred embodiment, the ASD of the present invention does not include any surfactant.

In another embodiment, the ASD of the present invention may include a surfactant to enhance solubility and/or to improve physical stability. A surfactant in general is in an amount of 5-40% w/w, preferably 10-30% w/w of the ASD.

A pharmaceutically acceptable surfactant useful as an additive in the solid dispersion may include polysorbate (e.g., polysorbate 20, polysorbate 40, polysorbate 80, polysorbate 85, polysorbate 60, etc.), cyclodextrin, polyoxyl 20 stearate, polyoxyl 35 castor oil, poloxamer, polyoxyethylene sorbitan monoisostearate, polyethylene glycol 40 sorbitan diisostearate, polyoxyl 40 hydrogenated castor oil, poloxamer 331, polyoxyethylene fatty acid esters, polyoxyl 40 castor oil, poloxamer 188, polyoxyethylene polyoxypropylene 1800, oleic acid, sodium desoxycholate, sodium lauryl sulfate, sorbitan monolaurate, sorbitan monooleate, sorbitan monopalmitate, sorbitan trioleate, N-carbamoyl methoxypolyethylene glycol 2000-1,2-distearol, myristic acid, steareth, stearic acid, polyoxyl 40 stearate, polyoxyl 60 stearate, sucrose stearate, tocopherol, polyoxyl castor oil, triglyceride synthetic, trimyristin, tristearin, magnesium stearate, lecithin, lauryl sulfate, vitamin E, egg yolk phosphatides, docusate sodium, dimyristoyl phosphatidylglycerol, dimyristoyl lecithin, Capryol 90 (propylene glycol monocaprylate), Capryol PGMC (propylene glycol monocaprylate), deoxycholate, cholesterol, Cremophor EL, Propylene glycol alginate, Croval A-10 (PEG 60 almond glycerides), Labrafil 1944 (oleoyl macrogol-6 glycerides), Labrafil 2125 (linoleoyl macrogol-6 glycerides), Labrasol (caprylocaproyl macrogol-8 glycerides), Lauroglycol 90 (propylene glycol monolaurate), Lauroglycol FCC (propylene glycol laurate), calcium stearate, Lecithin Centromix E, Lecithin Centrophase 152, Lecithin Centrol 3F21B, POE 26 glycerin, Olepal isosteariques (PEG-6 isostearate), Plurol diisostearique (polyglycerol-3-diisostearate), Plurol Oleique CC, POE 20 Sorbitan trioleate, Tagat TO (polyoxyethylene glycerol trioleate), or Solutol (Macrogol-15 hydroxystearate), or a mixture thereof.

The physical state of the ASD is analyzed with an X-ray powder diffractometer (XRPD). The results show that the ASD of Compound I does not have a crystalline form peak, which confirms Compound I being amorphous in ASD.

The thermochemical property of the ASD is analyzed with a differential scanning calorimeter (DSC). The results show that the ASD of Compound I has only one glass transition temperature and does not show any endothermic peak (melting peak), which confirms Compound I being amorphous in ASD. The resulting ASD can be formulated into pharmaceutical compositions that exhibit high bioavailability.

The ASD of the present invention provides a better solubility when dissolved in a pH 6.8 phosphate buffer than Type A crystalline.

The ASD of the present invention is stable and remains as amorphous for at least one month at 25-40° C. in 60-75% relative humidity.

In one embodiment, the ASD of the present invention is chemically stable and exhibits no significant purity change when stored at 25-40° C. in 60-75% relative humidity for at least one month.

### Methods for Preparing ASD

The ASD of the present invention may be prepared via spray drying, hot melt extrusion, or lyophilization technique.

The solid matrix has Compound I finely dispersed (molecular dispersion) in such a way that the solubility of the compound is maximized, thereby improving the bioavailability of the compound.

In one embodiment, the ASD of the present invention is prepared by dissolving Compound I in a sufficient amount of an organic solvent and mixing the resulting solution with a solution containing a pharmaceutically acceptable carrier and optionally a solubility enhancer such as a surfactant, resulting in a spray solution. The solvent may then be evaporated off, leaving the drug dispersed/dissolved in the matrix.

In one embodiment, the method comprises the steps of: (a) dissolving Compound I and a pharmaceutically acceptable carrier in a solvent; and (b) drying the solution obtained in step (a).

In one embodiment, step (a) comprises: dissolving Compound I in a sufficient amount of an organic solvent; dissolving a pharmaceutically acceptable carrier in a solvent; and then mixing the two solutions.

In one embodiment, organic solvents are used for dissolving Compound I and the carrier. The organic solvents may include an alcohol, a haloalkane, acetone, acetic acid, ethyl acetate, N,N-dimethylformamide, DMSO, tetrahydrofuran, or a mixture thereof. For example, the alcohol is methanol, ethanol, propanol, or isopropanol. For example, the haloalkane is dichloromethane, chloroform, or carbon tetrachloride.

In one embodiment, water or a mixture of water and an organic solvent is used for dissolving Compound I and the carrier.

In one embodiment, step (b) comprises spray drying. In another embodiment, step (b) comprises spray drying in combination with a fluid bed. In a further embodiment, step (b) comprises evaporation of the solvent using a rotovap.

In one embodiment, the solvent may be removed by evaporation via spray drying technique. The term “spray drying” being used conventionally and broadly refers to processes involving breaking up liquid mixture into small droplets (atomization) and rapidly removing solvent from the mixture in a spray-drying apparatus (e.g., a nozzle) where there is a strong driving force for evaporation of solvent from the droplets. In a typical spray drying process, the feed liquid may be a solution, slurry, emulsion, gel or paste, provided that it is pumpable and capable of being atomized.

Spray-drying processes and spray-drying equipment are described generally in Perry's Chemical Engineers' Handbook, pages 20-54 to 20-57 (Sixth Edition, 1984). The driving force for solvent elimination or evaporation is usually provided by keeping the partial pressure of solvent in the spray-drying equipment substantially below the vapor pressure of the solvent at the temperature of the drying droplets.

Once spraying is over, the feed and atomization are stopped, and the resulting solid dispersion is collected and vacuum-dried further if necessary in an oven at about 40-60° C.

In one embodiment, the ASD is prepared by hot-melt extrusion. In this method, Compound I and a carrier are first mixed uniformly. The mixture is fed to an extruder and extruded at a higher temperature than the melting temperature of the mixture of Compound I and the carrier. The collected solid is grinded and passed through a mesh filter to produce the ASD powder.

### Pharmaceutical Compositions

The present invention is also directed to a pharmaceutical composition comprising a therapeutically effective amount of Compound I in an ASD form and pharmaceutically acceptable excipient(s).

Pharmaceutically acceptable excipients, which are inactive ingredients, can be selected by those skilled in the art using conventional criteria. Pharmaceutically acceptable excipients include, but are not limited to, non-aqueous based solutions, suspensions, emulsions, microemulsions, micellar solutions, gels, and ointments. The pharmaceutically acceptable excipients may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate, and trolamine; antioxidants such as salts, acids and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositol; poloxamers and poloxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Such pharmaceutically acceptable excipients may be preserved against bacterial contamination using well-known preservatives, these include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet or a capsule formulation comprising Compound I may contain other excipients that have no bioactivity and no reaction with the active compound. Excipients of a tablet may include fillers, binders, lubricants and glidants, disintegrators, wetting agents, and release rate modifiers. Binders promote the adhesion of particles of the formulation and are important for a tablet formulation. Examples of binders include, but not limited to, carboxymethylcellulose, cellulose, ethylcellulose, hydroxypropylmethylcellulose, methylcellulose, karaya gum, starch, and tragacanth gum, poly(acrylic acid), and polyvinylpyrrolidone.

In one embodiment the composition is tableted.

In one embodiment, a tablet formulation comprises ASD of the present invention in a weight range of 5-75%, preferably 5-40%.

In one embodiment, a tablet formulation comprises one or more fillers, for example lactose and/or microcrystalline cellulose, in a total weight percent range of 10-80%, preferably 20-80% or 40-80%.

In one embodiment, a tablet formulation comprises a disintegrant in a weight percentage of 4-20%, preferably 5-15%.

In one embodiment, a tablet formulation comprises a glidant, in a weight percent range of 0.5-10%, preferably 1-5%. In one embodiment, a tablet formulation comprises a lubricant, for example, magnesium stearate, in a weight percent range of 0.25-2.0%, preferably 0.25-1.0%, and more preferably 0.5-1.0%.

The following examples further illustrate the present invention. These examples are intended merely to be illustrative of the present invention and are not to be construed as being limiting.

### EXAMPLES

### Example 1. Preparation of Type A Crystalline Form

Compound I was prepared according to the procedures described in Example 21 of WO2018/049781. Compound I was first dissolved in a mixed solvent of acetone and deionized water (4:1 v/v) by heating to 50° C. and the temperature of the resulting solution was then decreased to 30° C., followed by addition of crystal seed. The temperature of the mixture was further lowered to 20-25° C. for precipitation. The solid was filtered and washed with a mixed solvent of acetone/water (2:3 v/v) to provide Type A crystalline.

### Example 2. Preparation of ASD via Spray Drying

HPMCAS-10-SDD (10% drug loading): 2.00 g of Compound I was dissolved in 360 mL of methanol/acetone (4:6 v/v). While stirring at 100 rpm, 18.00 g of HPMC-AS was added slowly. After addition, stirring was continued for 2 hours until a clear solution was obtained. The solution was then placed in a Yamato ADL 311S spray-dryer for ASD preparation which was set with inlet temperature of 80° C., outlet temperature of 46-52° C., flow-rate of 5.2-11.3 mL/min and spray air pressure of 0.1 MPa. The collected spray-dried material was transferred to a glass dish, covered and vacuum-dried for 2 hours to give 15.06 g ASD powder.

Table 2 show four ASDs prepared according to the above-described procedure with different amounts of Compound I and reagents.

TABLE 2

| | | | | HPMCAS/ |
|---|---|---|---|---|
| | HPMCAS- | HPMCAS- | HPMCAS- | Poloxamer- |
| Sample | 10-SDD | 20-SDD | 40-SDD | 10-SDD |
| Drug | 10 | 20 | 40 | 10 |
| loading (%) | | | | |
| Compound | 2.00 | 2.51 | 5.00 | 1.50 |
| I (g) | | | | |
| HPMC-AS (g) | 18.00 | 10.00 | 7.51 | 10.50 |
| Poloxamer (g) | — | — | — | 3.00 |
| Methanol/ | 360 | 180 | 360 | 270 |
| acetone* | | | | |
| (4:6 v/v) | | | | |
| (mL) | | | | |
| ASD (g) | 15.06 | 9.88 | 9.34 | 10.78 |

*Methanol and acetone were removed during ASD preparation.

### Example 3. Preparation of ASD via Hot Melt Extrusion

PVPVA-10-HME (10% drug loading): 2.00 g of Compound I and 18.00 g of PVP-VA were mixed uniformly. After passing through a 40-mesh filter, the mixture was fed to a Thermo MiniCTW extruder for ASD preparation at 20 rpm under 160° C. The collected yellow solid was grinded and passed through a 40-mesh filter to give 11.35 g ASD powder. The extrusion was carried out at 30 rpm under 180° C.

PVPVA-20-HME (20% drug loading) and PVPVA-40-HME (40% drug loading) were prepared similarly with different amounts of PVP-VA (see Table 3).

TABLE 3

| | PVPVA- | PVPVA- | PVPVA- |
|---|---|---|---|
| Sample | 10-HME | 20-HME | 40-HME |
| Drug loading (%) | 10 | 20 | 40 |
| Compound I (g) | 2.00 | 2.00 | 4.00 |
| PVP-VA (g) | 18.00 | 8.00 | 6.01 |
| ASD (g) | 11.35 | 5.16 | 5.05 |

### Example 4. Solubility Profiles of Type A and ASD

Phosphate buffer (pH 6.8): 3.40 g of potassium dihydrogen phosphate and 0.45 g of sodium hydroxide were dissolved in 500 mL of water.

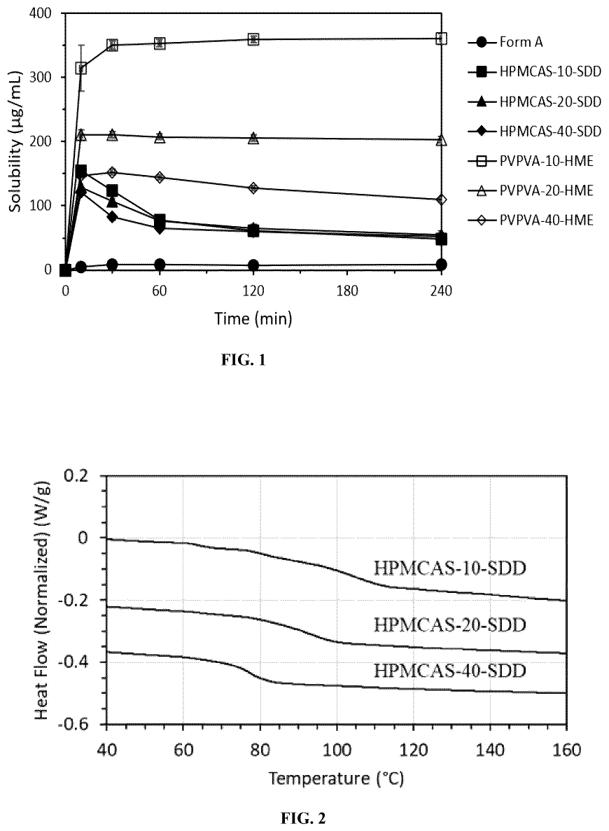

Type A: About 8.0 mg of Type A sample was added to 20 mL of the phosphate buffer (pH 6.8) in a 30-mL glass vial. The vial was capped, placed in a shaker, and kept shaking with 150 rpm at 37° C. At indicated time intervals (10, 30, 60, 120, 240 min), a fraction of the suspension was removed and filtered through a 0.45-μm filter by discarding the first part of the filtrate. The filtrate was diluted and then analyzed by HPLC. The results (averaged from three repeats) are shown in Table 4 and FIG. 1.

ASD: An appropriate amount of ASD (about 3.0 mg of Compound I) was added to 7.5 mL of the phosphate buffer (pH 6.8) in a 30-mL glass vial. The vial was capped, placed in a shaker, and kept shaking with 150 rpm at 37° C. At indicated time intervals (10, 30, 60, 120, 240 min), a fraction of the suspension was removed and centrifuged at 12,000 rpm for 5 minutes to give a clear solution that was then analyzed by HPLC. The results (averaged from three repeats) are shown in Table 4 and FIG. 1.

TABLE 4
Results of Solubilities of Type A and ASD in pH 6.8 Phosphate Buffer (n = 3)

| | | | Drug | Solubility (μg/mL) | | | | |
|---|---|---|---|---|---|---|---|---|
| | ASD | Method of | Loading | 10 | 30 | 60 | 120 | 240 |
| Sample | Carrier | Preparation | (%) | min | min | min | min | min |
| Type A | — | — | 100 | 4.88 | 7.91 | 7.81 | 7.62 | 8.47 |
| HPMCAS- | HPMC-AS | Spray | 10 | 154.75 | 123.67 | 77.47 | 61.02 | 48.78 |
| 10-SDD | | drying | | | | | | |
| HPMCAS- | | | 20 | 129.16 | 107.04 | 75.84 | 64.97 | 55.04 |
| 20-SDD | | | | | | | | |
| HPMCAS- | | | 40 | 120.52 | 82.13 | 65.03 | 59.22 | 52.26 |
| 40-SDD | | | | | | | | |
| PVPVA- | PVP-VA | Hot melt | 10 | 314.27 | 350.68 | 353.49 | 359.61 | 360.30 |
| 10-HME | | extrusion | | | | | | |
| PVPVA- | | | 20 | 210.37 | 210.29 | 207.46 | 205.55 | 203.34 |
| 20-HME | | | | | | | | |
| PVPVA- | | | 40 | 146.26 | 152.43 | 144.28 | 127.44 | 109.86 |
| 40-HME | | | | | | | | |

The results show that solubility increased significantly after Type A was converted into ASD with HPMCAS or PVPVA. As the drug loading increased, the solubility of ASD decreased and the decrease is more pronounced for ASD with PVPVA prepared via hot melt extrusion.

### Example 5. Glass Transition Temperature Determination

Type A: Accurately weighted Type A sample (2˜5 mg) was placed in a DSC pan which was then sealed and placed in TA Instruments Q2000. The pan was heated from room temperature to 230° C. at a rate of 20° C./min and after 5 min, rapidly cooled to 0° C. at a rate of 50° C./min. After another 5 min, the DSC analysis was carried out between 0 to 210° C. at a rate of 10° C./min. The glass transition temperatures (Tg) are summarized in Table 5.

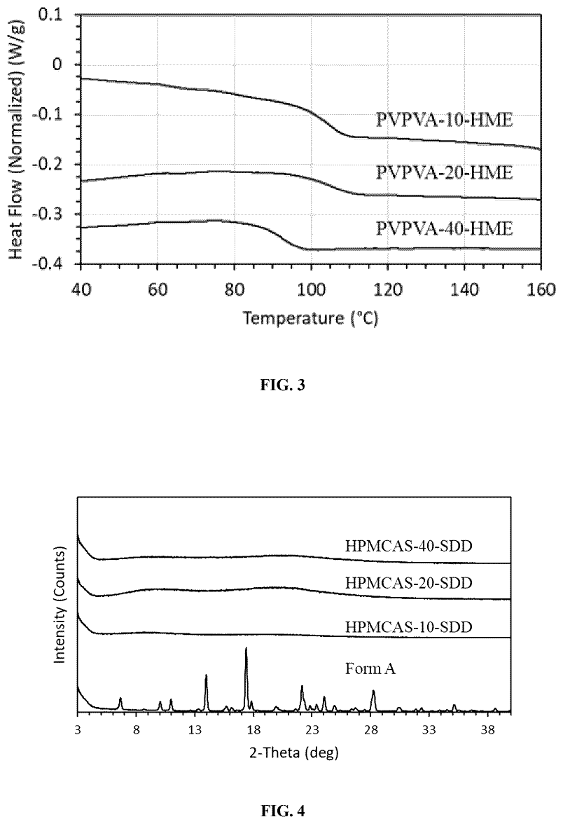

ASD: Accurately weighted ASD sample (2˜5 mg) was placed in a DSC pan which was then sealed and placed in TA Instruments Q2000. The pan was heated from room temperature to 135° C. at a rate of 20° C./min and after 5 minutes, rapidly cooled to 0° C. at a rate of 50° C./min. After another 5 min, the DSC analysis was carried out between 0 to 210° C. at a rate of 10° C./min (FIG. 2 and FIG. 3). The glass transition temperatures (Tg) are summarized in Table 5.

TABLE 5
Results of Glass Transition Temperature

| | ASD | Preparation | Drug Loading | Tg |
|---|---|---|---|---|
| Sample | Carrier | Method | (%) | (° C.) |
| Type A | — | — | 100 | 70.1 |
| HPMCAS-10-SDD | HPMC-AS | Spray drying | 10 | 106.2 |
| HPMCAS-20-SDD | | | 20 | 92.3 |
| HPMCAS-40-SDD | | | 40 | 77.4 |
| PVPVA-10-HME | PVP-VA | Hot melt | 10 | 104.4 |
| PVPVA-20-HME | | extrusion | 20 | 104.5 |
| PVPVA-40-HME | | | 40 | 92.2 |

DSC curves show that there is only one glass transition temperature without observed melting peak, indicating that Compound I exists in an amorphous state among carriers made by both techniques. When the percentage of drug loading increases from 10% to 40%, Tg decreases, therefore the risk of phase separation increases. It would be desirable to keep drug loading low to ensure physical stability.

### Example 6. XRPD

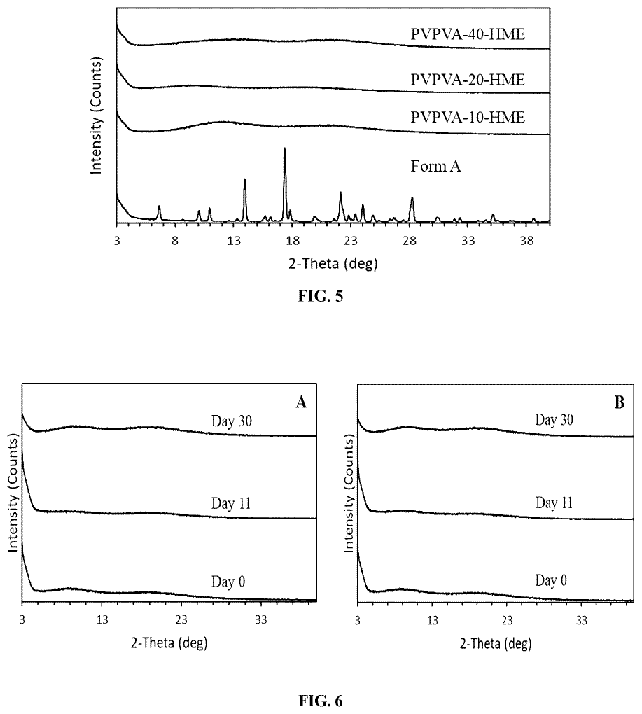

About 10 mg of each Type A crystalline and ASD was each sprayed on a silicon chip for XRPD measurement under 40 mA current, 40 kV voltage, 5˜40° 2θ, 0.0263° scanning step and 0.3282°/min scanning speed. The results are shown in FIG. 4 (HPMCAS) and FIG. 5 (PVPVA). Compound I in the ASD prepared by either spray drying or hot melt extrusion was shown in an amorphous state. XRPD shows no crystal diffraction peak in ASD powder, but characteristic diffraction peaks in Type A crystalline.

### Example 7. Hygroscopicity of ASD with 10% Drug Loading under High Humidity Condition

The ASDs of HPMCAS-10-SDD and PVPVA-10-HME were transferred to brown glass vials uncapped respectively, which were then placed inside desiccators to investigate the hygroscopicity. The desiccators contained a saturated potassium nitrate solution at the bottom to maintain 92.5% relative humidity (RH). The ASDs were weighed after 0 and 10 days and inspected for their appearances. The weights increases were calculated as shown in Table 6. After 10 days under the high humidity condition, HPMCAS-10-SDD solid dispersion remained as a while or almost white powder, without obvious change in the appearance, and had low degrees of hygroscopicity. On the contrary, PVPVA-10-HME solid dispersion had certain appearance change from an almost white powder to transparent semi-solid and significant weigh increase due to moisture absorption, indicating its high hygroscopicity, which is related to high hygroscopic nature of the carrier PVP-VA.

TABLE 6
Results of Weight Increase due to Moisture Absorption

| | | Day 10 under 92.5% RH | | |
|---|---|---|---|---|
| | Day 0 | Moisture- | Ratio of | Average |
| | Drug | absorption | moisture- | of weight |
| | weight | weight | absorption | gain ratio |
| Sample | (mg) | gain (mg) | weight gain | (%) |
| HPMCAS- | 203.7 | 10.9 | 5.35 | 5.24 |
| 10-SDD | 204.9 | 10.5 | 5.12 | |
| PVPVA- | 212.0 | 28.3 | 13.35 | 12.27 |
| 10-HME | 202.7 | 22.7 | 11.20 | |

### Example 8. Stability Assessment of ASD

Capped brown glass vials each containing an ASD sample (HPMCAS-10-SDD and PVPVA-10-HME) were placed inside stability chambers. After 11 and 30 days under 25° C./60% RH and 40° C./75% RH, the samples were evaluated by XRPD, DSC, solubility, and relative substances, respectively.

After stored under 40° C./75% RH for 11 and 30 days, the appearance of PVPVA-10-HME ASD changed significantly, from an almost white powder to transparent semi-solid glued to the inner wall of the brown bottle, which is not acceptable. The samples remained glued to the wall during shaking, therefore no sample was taken for measurement. HPMCAS-10-SDD ASD appeared normal after stored under the same condition for 11 and 30 days, further showing that HPMCAS-10-SDD had better physical and chemical stabilities, and met product specification.

### 1) XRPD Results

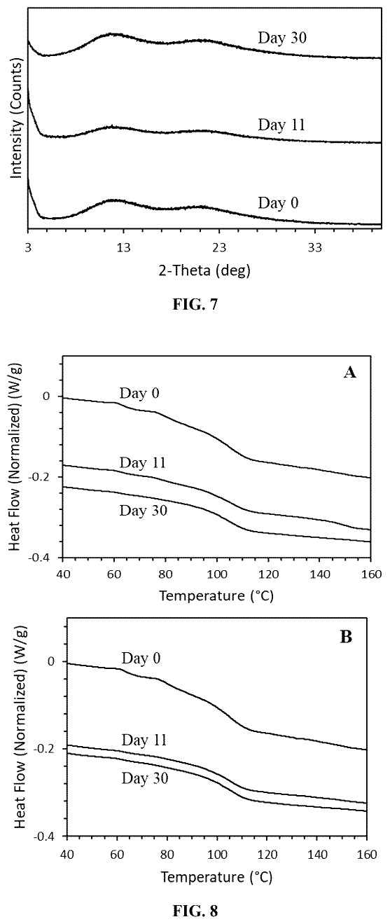

The XRPD diagrams of HPMCAS-10-SDD after stored under (A) 25° C./60% RH, sealed and (B) 40° C./75% RH, sealed for 0-30 days are shown in FIG. 6. The XRPD diagrams of PVPVA-10-HME stored under 25° C./60% RH, sealed for 0-30 days are shown in FIG. 7. The results show that there is no observed crystalline diffraction peak in the ASD samples, indicating that the ASD samples remain as amorphous at least for 30 days.

### 2) DSC Results

The DSC curves of HPMCAS-10-SDD after stored at (A) 25° C./60% RH, sealed and (B) 40° C./75% RH, sealed for 0-30 days are shown in FIG. 8. The results show that only one Tg is observed without extra melting peak, indicating that the ASD samples remain as amorphous at least for 30 days.

### 3) Solubility Results

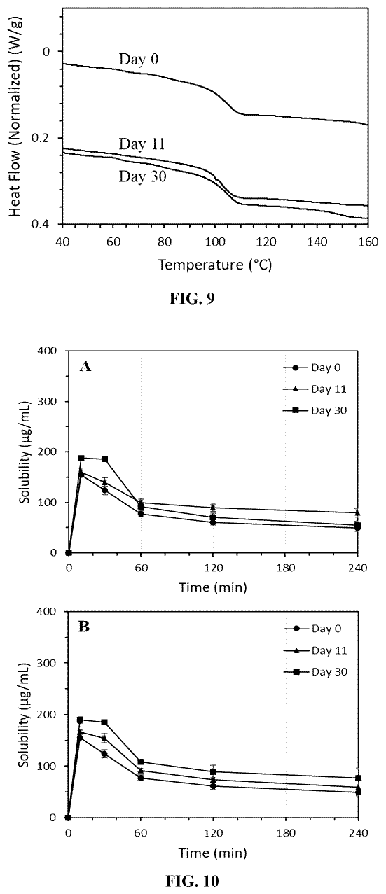

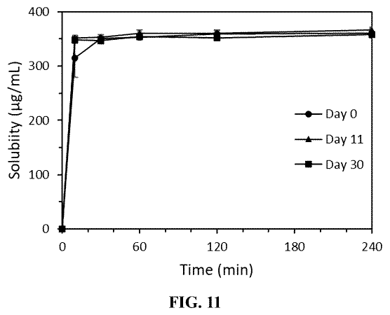

FIG. 10 shows the solubility curves of HPMCAS-10-SDD (in a pH 6.8 phosphate buffer medium, n=3) after stored under (A) 25° C./60% RH, sealed and (B) 40° C./75% RH, sealed for different time periods (0, 11 and 30 days). FIG. 11 shows the solubility curves of PVPVA-10-HME (in a pH 6.8 phosphate buffer media, n=3) after stored under 25° C./60% RH, sealed for different time periods (0, 11, 30 days). The results show that there is no obvious change in the solubility profiles for two ASD samples after stored under two conditions for 30 days.

### 4) Relative Substance Results

Relative substance evaluations of HPMCAS-10-SDD after stored under (A) 25° C./60% RH, sealed and (B) 40° C./75% RH, sealed, and PVPVA-10-HME after stored under 25° C./60% RH, sealed for different time periods (0, 11 and 30 days) are summarized in Table 7. The results show that there was no obvious change in relative substance for two ASD samples after stored under two conditions for 11 and 30 days; both sample had acceptable stability.

TABLE 7
Results of Relative Substances of
HPMCAS-10-SDD and PVPVA-10-HME

| | Storage | Time | Total |
|---|---|---|---|
| Sample | Condition | (day) | Impurities (%) |
| Type A | — | — | 0.19 |
| HPMCAS-10-SDD | 25° C./60% RH | 0 | 0.19 |
| | sealed | 11 | 0.19 |
| | | 30 | 0.24 |
| | 40° C./75% RH | 0 | 0.19 |
| | sealed | 11 | 0.25 |
| | | 30 | 0.26 |
| PVPVA-10-HME | 25° C./60% RH | 0 | 0.26 |
| | sealed | 11 | 0.25 |
| | | 30 | 0.26 |

### Example 9. In Vivo PK

Type A and HPMCAS-10-SDD were formulated with excipients, respectively. The PK properties of two formulations administered orally in beagle dogs were evaluated. Each formulation was tested in three dogs, each weighed 8˜12 kg.

### 1) In Vivo PK Determination of Type A Formulation

A formulation was prepared by wet granulation. Firstly, mannitol, pregelatinized starch, sodium carboxymethyl starch and sodium dodecyl sulfate of different amounts were mixed evenly according to Table 8. Secondly, Type A and Povidone K30 were added into an appropriate amount of absolute alcohol, stirred, and mixed as adhesive. Finally, the adhesive was added to the evenly mixed excipients, followed by granulation, drying, addition of magnesium stearate, and mixing into a powder.

TABLE 8
The Formulation of Type A

| Composition | Amount (mg) |
|---|---|
| Drug substance (Type A) | 5 |
| mannitol | 265 |
| Pregelatinized starch | 56.25 |
| Sodium carboxymethyl starch | 30 |
| Sodium dodecyl sulfate | 7.5 |
| Povidone K30 | 7.5 |
| Absolute alcohol | ~0.06 mL |
| Magnesium stearate | 3.75 |
| Total | 375 |

The above formulation was added to 0.5% methylcellulose (MC) to give a suspension containing 0.6 mg/mL of Compound I. The suspension was then orally administered to fasted beagle dogs at 5 mL/kg, which was equivalent to a dose of 3 mg/kg. Blood samples were collected at 0.25, 0.5, 1, 2, 4, 8 and 24 hours post dose. Concentrations of Compound I were quantified by LC-MS/MS analysis using an API-4500 mass spectrometer. The PK parameters were calculated using WinNonlin and the results are summarized in Table 10.

### 2) In Vivo PK Determination of HPMCAS-10-SDD Formulation

A formulation was prepared by dry granulation. HPMCAS-10-SDD, mannitol, microcrystalline cellulose, different amounts of pregelatinized starch and silicon dioxide were mixed evenly according to Table 9, followed by dry granulation, addition of magnesium stearate, and tableting.

TABLE 9
Formulation of HPMCAS-10-SDD

| Composition | Amount/tablet (mg) |
|---|---|
| HPMCAS-10-SDD | 80 |
| Mannitol | 482 |
| Microcrystalline cellulose | 96 |
| Pregelatinized starch | 120 |
| Silicon dioxide | 16 |
| Magnesium stearate | 6 |
| Total | 800 |

Four tablets were orally administered to fasted beagle dogs at a dose of 3 mg/kg. Blood samples were collected at 0.25, 0.5, 1, 2, 4, 8 and 24 hours post dose. Concentrations of Compound I were quantified by LC-MS/MS analysis. The PK parameters were calculated using WinNonlin and the results are summarized in Table 10.

TABLE 10
In vivo Dog PK Results

| | Dosage | | Cₘₐₓ | AUCₗₐₛₜ |
|---|---|---|---|---|
| Sample | (mg/kg) | Formulation | (ng/mL) | (h*ng/mL) |
| Type A | 3 | 0.5% MC | 270 | 1252 |
| | | suspension | | |
| HPMCAS-10-SDD | 3 | Tablet | 886 | 2733 |

The results show that HPMCAS-10-SDD has better Cₘₐₓ and AUC in beagle dogs than Type A.

It is to be understood that the foregoing describes preferred embodiments of the present invention and that modifications may be made therein without departing from the scope of the present invention as set forth in the claims.

What is claimed is:

1. An amorphous solid dispersion comprising 5-40% w/w of compound I or a pharmaceutically acceptable salt thereof, and 60-95% w/w of hydroxypropylmethylcellulose-acetate succinate (HPMC-AS)

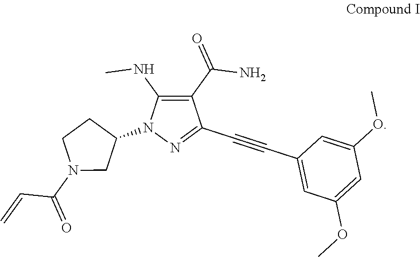 [SMILES: C=CC(=O)N1CC[C@H](n2nc(C#Cc3cc(OC)cc(OC)c3)c(C(N)=O)c2NC)C1]

2. The amorphous solid dispersion according to claim 1, comprising 5-20% w/w of compound I.

3. The amorphous solid dispersion according to claim 1, comprising 10% w/w of compound I.

4. The amorphous solid dispersion according to claim 1, prepared by either spray-drying or hot-melt extrusion.

5. A pharmaceutical composition comprising the amorphous solid dispersion according to claim 1 and a pharmaceutically acceptable carrier.

6. The pharmaceutical composition of claim 5, in a tablet form or a capsule form.

7. A method for preparing the amorphous solid dispersion of claim 1, comprising the steps:
(a) dissolving compound I or a pharmaceutically acceptable salt thereof and HPMC-AS in an organic solvent to form a solution; and
(b) spray-drying the solution to form the amorphous solid dispersion.
